(19) United States
(12) Patent Application Publication
(10) Pub. No.: US 2017/0112650 A1
(43) Pub. Date: Apr. 27, 2017

(54) MAGNETIC DEVICES AND RELATED METHODS FOR GASTROINTESTINAL RESTRICTION

(71) Applicants: Boston Scientific Scimed, Inc., Maple Grove, MN (US); Lifespan Corporation, Providence, RI (US)

(72) Inventors: John A. HINGSTON, Framingham, MA (US); Gene T. STORBECK, Franklin, MA (US); Jeri Ann HILLER, Westford, MA (US); Kathleen CORCORAN, Watertown, MA (US); Selina M. MERKLING, St. Louis Park, MN (US); Kali L. MANNING, Providence, RI (US); Gary Dean ROYE, Cranston, RI (US); Sivamainthan VITHIANANTHAN, Sharon, MA (US); Beth RYDER, Warwick, RI (US)

(21) Appl. No.: 15/333,720

(22) Filed: Oct. 25, 2016

Related U.S. Application Data
(60) Provisional application No. 62/246,930, filed on Oct. 27, 2015.

(51) Int. Cl. A61F 5/00

(52) U.S. Cl. CPC A61F 5/0086; A61F 5/0069; A61F 5/0036; A61F 2210/0004; A61F 2210/009; A61F 2002/045

(57) ABSTRACT

A system for restricting a stomach of a patient may include a plurality of discrete magnetic devices. Each magnetic device may include a magnet and be configured to be secured to tissue. A first portion of the plurality of magnetic devices may be configured to be transorally inserted into the patient and secured to a first wall of the stomach, and a second portion of the plurality of magnetic devices may be configured to be transorally inserted into the patient and secured to a second wall of the stomach. The first and second portions may be configured to be magnetically joined together to draw the first and second walls of the stomach towards each other.

### CROSS-REFERENCE TO RELATED APPLICATION

This application claims the benefit of priority under 35 U.S.C. §119 to U.S. Provisional Patent Application No. 62/246,930, filed Oct. 27, 2015, the entirety of which is incorporated herein by reference.

### TECHNICAL FIELD

The present disclosure relates generally to magnetic devices and related methods for altering an anatomical configuration of the gastrointestinal tract of a patient, particularly of the patient's stomach.

### BACKGROUND

Current bariatric (obesity treatment) procedures may reduce the size of the stomach or create restrictions in the stomach to promote weight loss. These procedures may be carried out laparoscopically (i.e., through small incisions in the skin) or during open surgery. For example, a sleeve gastrectomy procedure includes suturing or stapling the stomach longitudinally from the antrum to the fundus and removing a large portion of the stomach so that the stomach forms a “sleeve.” This procedure is irreversible. In another procedure, a gastric band is placed around the exterior of the stomach to create a restriction that limits food consumption.

Even if performed laparoscopically, current bariatric procedures may be relatively invasive in that tissue is cut and removed, leading to a risk of infection. In a sleeve gastrectomy, the staple line may leak. Furthermore, if a line of sutures or staples restricts a portion of a patient's stomach and too much pressure is exerted on the restriction, the line may open up or the stomach tissue may rip.

### SUMMARY

Examples of the present disclosure relate to, among other things, magnetic devices and methods for using the magnetic devices to alter an anatomical configuration of a patient's gastrointestinal system. Each of the examples disclosed herein may include one or more of the features described in connection with any of the other disclosed examples.

In one example, a system for restricting a stomach of a patient may include a plurality of discrete magnetic devices. Each magnetic device may include a magnet and be configured to be secured to tissue. A first portion of the plurality of magnetic devices may be configured to be transorally inserted into the patient and secured to a first wall of the stomach, and a second portion of the plurality of magnetic devices may be configured to be transorally inserted into the patient and secured to a second wall of the stomach. The first and second portions may be configured to be magnetically joined together to draw the first and second walls of the stomach towards each other.

Additionally or alternatively, any system described herein may include one or more of the following features: the magnetic devices may be configured to be received within a delivery device during transoral insertion; each magnetic device may include a bioabsorbable anchor; the first and second portions may include a same number of magnetic devices; each magnet may include an opening for receiving a portion of the anchor; each anchor may include a central elongated portion and at least two legs, the central elongated portion may be configured to couple to the magnet, and the two legs may be configured to pass through the respective stomach wall to secure the magnet to the respective stomach wall; the anchor may include a suture; each magnet may be configured to be secured to an interior of the stomach; when magnetically joined, the first and second portions may be configured to alter a flow of food within the stomach; each magnet may include at least one of a circular shape or an elongated rectangular shape; each magnet may include a therapeutic coating; when magnetically joined, the first and second portions may be configured to reduce an effective volume of the stomach; each magnet may be a permanent magnet; when magnetically joined, the first and second portions may form a plurality of magnetically-joined pairs of magnets, each pair including a magnet of the first portion and a magnet of the second portion; and the plurality of magnetically-joined pairs of magnets may be configured to be spaced apart from each other.

In one example, a method of restricting a stomach of a patient may include transorally inserting a first plurality of magnets and a second plurality of magnets into the stomach of the patient, wherein the magnets of the first plurality are magnetically attracted to the magnets of the second plurality; securing each magnet of the first plurality of magnets to a discrete location on an interior of a first wall of the stomach, and securing each magnet of the second plurality of magnets to a discrete location on an interior of a second wall of the stomach; and allowing a plurality of pairs of magnets, each pair including a magnet of the first plurality of magnets and a magnet of the second plurality of magnets, to magnetically join together to create a restriction in the stomach.

Additionally or alternatively, any method described herein may include one or more of the following features or steps: each magnet of the first plurality of magnets and each magnet of the second plurality of magnets may be secured to the respective wall of the stomach by a different bioabsorbable anchor; the plurality of pairs are arranged in a curved configuration extending from a proximal end adjacent a fundus to a distal end closer to an antrum than the proximal end; the method may further comprise providing a distal space between the distal end and a wall of the antrum, wherein the distal space is greater than a proximal space between the proximal end and a wall of the fundus; the magnetically-joined plurality of pairs of magnets may at least partially block food from a region of the stomach that includes the fundus and the greater curvature; the plurality of magnetically-joined pairs of magnets may be configured to allow at least some food to pass between adjacent pairs of magnets; the plurality of pairs may be arranged in a linear configuration extending from a first end adjacent a wall of a lesser curvature of the stomach to a second end closer to a greater curvature of the stomach than the first end; the method may further comprise providing a first space between the second end and the greater curvature, wherein the first space is greater than a second space between the first end and the wall of the lesser curvature; and the plurality of pairs may be arranged in a curved configuration extending from a first end adjacent a proximal wall of a lesser curvature of the stomach and a second end adjacent a distal wall of the lesser curvature.

In another example, a method of restricting a stomach of a patient may include transorally inserting a plurality of magnets into the stomach of the patient; and securing each magnet of the plurality of magnets to an interior of the stomach with an anchor, wherein each magnet is secured with a different anchor; and wherein a first portion of the plurality of magnets is secured to a first wall of the stomach, and a second portion of the plurality of magnets is secured to a second wall of the stomach, and the first and second portions are configured to magnetically join to create a restriction that guides food from a distal end of an esophagus towards a fundus.

Additionally or alternatively, any method described herein may further comprise one or more of the following features or steps: the first and second portions may be configured to separate to relieve pressure in the stomach; the restriction may extend in a linear configuration from a first end adjacent a wall of a lesser curvature of the stomach to a second end closer to a greater curvature of the stomach than the first end; the restriction may extend in a curved configuration from a first end adjacent a proximal wall of a lesser curvature of the stomach to a second end adjacent a distal wall of the lesser curvature; the restriction may be configured to allow at least some food to pass through the restriction; and each magnet of the plurality of magnets may include a circular shape.

In yet another example, a method of restricting a stomach of a patient may include inserting a plurality of discrete magnets through an esophagus and into the stomach of the patient; and securing each of the plurality of magnets to a wall of the stomach, wherein each magnet is magnetically attracted to at least one other magnet; wherein the magnets are arranged in at least a first group and a second group, with adjacent magnets in the first group spaced apart from each other and with adjacent magnets in the second group spaced apart from each other, and wherein the first group and the second group are configured to move towards each other through magnetic attraction and to separate from each other to relieve a pressure in the stomach.

Additionally or alternatively, any method described herein may include one or more of the following features or steps: each of the plurality of discrete magnets may be secured to an interior of the stomach; the first group may include the same number of magnets as the second group; each magnet of the plurality of magnets may be secured to the wall of the stomach by an anchor that is configured to release the magnet from the wall of the stomach after a predetermined period of time; and when the first group and the second group move towards each other, the stomach may be divided into a first elongated region for a flow of food, the first elongated region having a proximal end adjacent an esophagus and a distal end adjacent an intestine, and a second region at least partially blocked from the flow of food.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

### BRIEF DESCRIPTION OF THE DRAWINGS

The accompanying drawings, which are incorporated in and constitute a part of this specification, illustrate examples of the present disclosure and together with the description, serve to explain the principles of the disclosure.

FIG. 1 illustrates a perspective view of a magnetic device.

FIG. 2 illustrates a side view of a pair of magnetic devices, with each magnet secured to gastrointestinal wall tissue.

FIGS. 3A, 3B, and 3C illustrate a method for securing a magnetic device to gastrointestinal wall tissue.

FIG. 4 illustrates a cross-sectional view of a patient's stomach showing a first exemplary arrangement of magnets.

FIG. 5 illustrates the view of the first arrangement shown in FIG. 4, with magnets having a different shape.

FIG. 6 illustrates a cross-sectional view of a patient's stomach showing a second exemplary arrangement of magnets.

FIG. 7 illustrates a cross-sectional view of a patient's stomach showing a third exemplary arrangement of magnets.

FIG. 8 illustrates a cross-sectional view of a patient's stomach showing a fourth exemplary arrangement of magnets.

### DETAILED DESCRIPTION

The present disclosure is drawn to magnetic devices and methods for using the magnetic devices to alter an anatomical configuration of a patient's gastrointestinal system, particularly of the patient's stomach. A surgeon may implant the magnetic devices in a variety of arrangements to alter the patient's anatomy. The magnetic devices may be implanted endoscopically. The procedures disclosed herein may be less invasive than some existing bariatric procedures. In addition, once implanted, the devices described herein may minimize post-procedure complications because individual magnets may temporarily separate from each other to relieve excess pressure.

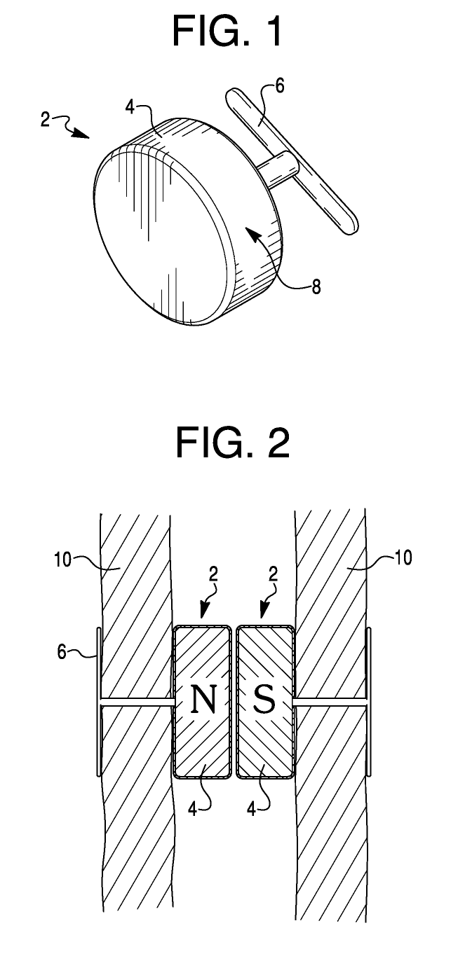

Referring to FIG. 1, a magnetic device 2 may include a magnet 4 and an anchor 6. The magnet 4 may be any type of magnet that is capable of being magnetically attracted to another magnet 4. For example, the magnet 4 may be a permanent magnet, a material that can be magnetized, or an electromagnet. The magnet 4 may be any shape, such as circular, oval, rectangular, square, or irregularly shaped. The magnet 4 may include throughholes or surface irregularities to promote tissue ingrowth. The magnet 4 may include a coating 8. The coating 8 may be biocompatible and may be resistant to stomach fluids. For example, the coating 8 may be silicone, liquid silicone rubber, polytetrafluoroethylene (PTFE), polyethylene terephthalate (PET), ultra-high-molecular-weight polyethylene (UHMWPE), or any biomaterial resistant to the acidic environment within the stomach. In one example, the coating 8 may be therapeutic. For example, the coating 8 may include antibacterial properties, or may include hormones or other compounds designed to release, trigger the release, or reduce the release of hormones that affect feelings of satiety (e.g., ghrelin, leptin, or cholecystokinin (CCK)).

The anchor 6 may be coupled to the magnet 4 and may secure the magnet 4 to tissue within a patient. In one example, the anchor 6 is a t-tag. Additionally or alternatively, the anchor 6 may include a suture, loop, or other device configured to attach the magnet 4 to tissue. For example, the anchor 6 may include a pouch that encloses the magnet 4 and is stapled or sutured to tissue. In one example, the anchor 6 may be bioabsorbable. As used herein, the term bioabsorbable means any material that will degrade over time in the body and includes biodegradable and bioresorbable materials. The material of the anchor 6 may be chosen to degrade over a certain period of time to place a time limit on the restrictive effects of the magnetic devices 2. Once the anchor 6 degrades sufficiently, the magnet 4 may be released from tissue, such as the wall of the stomach, and may pass through the patient's digestive system. The anchor 6 may be configured to degrade after a predetermined period of time, such as two years, for example. The patient then may return to his or her native anatomy. In one example, the magnets 4 do not have sharp corners or edges, allowing the magnets to pass through the patient with a reduced risk of trauma to the gastrointestinal tract.

FIG. 2 illustrates a pair of magnetic devices 2 secured to stomach wall tissue 10. In one example, a first magnetic device 2 is secured to a first wall of a stomach (e.g., a posterior wall) and a second magnetic device 2 is secured to a second wall of the stomach (e.g., an anterior wall). Once secured to tissue 10, the magnets 4 of the magnetic devices 2 may be in an interior of the stomach, with the anchors 6 passing through the stomach wall tissue 10. A portion of the anchor 6 may be exterior to the stomach. The magnetic devices 2 magnetically attract each other, and once secured to the tissue 10, may pull the stomach walls towards each other to create a restriction.

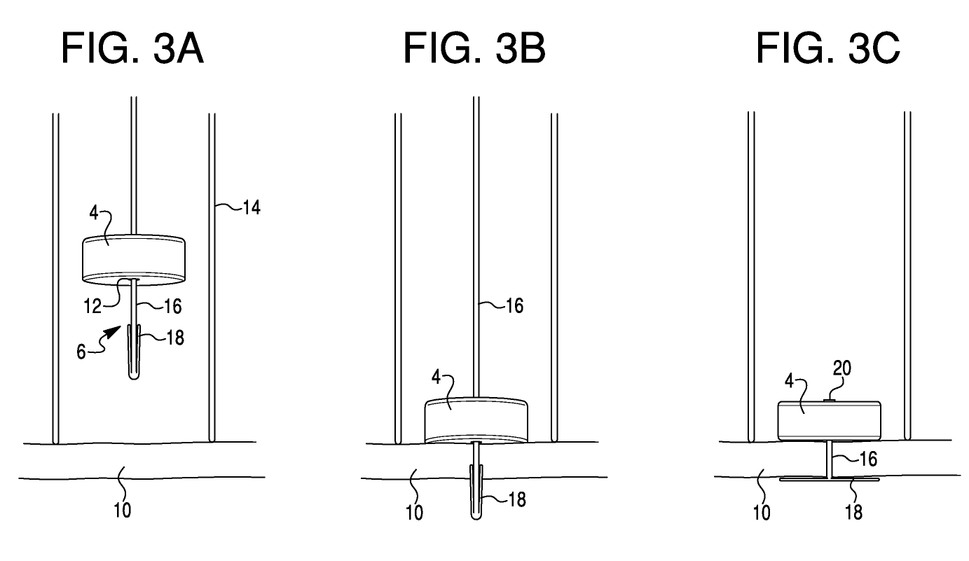

The magnetic devices 2, including magnets 4 and corresponding anchors 6, may be inserted transorally and passed through the esophagus and into the patient's stomach. During the transoral insertion step, the magnetic devices 2 may be contained within a delivery device having a distal end 14 (see FIGS. 3A-3C). An endoscope may be used to visualize insertion of the magnetic devices 2 and the attachment of the devices 2 to the stomach wall tissue 10. The magnetic devices 2 may be inserted into the patient one at a time and secured to the patient's stomach one at a time. Alternatively, several magnetic devices 2 may be inserted into the patient's stomach at one time but then secured to stomach wall tissue 10 one at a time. In one example, each magnetic device 2 may be secured to a discrete location on the stomach wall, with no connection to adjacent magnetic devices 2.

FIGS. 3A-3C illustrate an exemplary method of securing a magnetic device 2 to stomach wall tissue 10. As shown in FIG. 3A, the anchor 6 in this example includes a t-tag having a central elongated portion 16 and two legs 18. The central elongated portion 16 may be placed through an opening 12 in the magnet 4, with the legs 18 in a collapsed configuration. A vacuum may be created in the distal end 14 of the device to hold the stomach wall taut. In addition, the vacuum may be used to pull the stomach wall toward the interior of the stomach and away from any tissue on the opposite side of the stomach wall. This may prevent the tissue on the opposite side of the stomach wall from being penetrated by the t-tag.

The delivery device may be used to place the legs 18 of the t-tag across the stomach wall tissue 10. As shown in FIG. 3B, the legs 18 of the anchor 6 are pushed through the stomach wall tissue 10. Once through the stomach wall tissue 10, the legs 18 may expand as shown in FIG. 3C. The legs 18 may be biased to the expanded configuration shown in FIG. 3C. A sheath or a vacuum may hold the legs 18 in the collapsed configuration during the step illustrated in FIG. 3A. Once the legs 18 are expanded as shown in FIG. 3C, the device used to implant the anchors 6 may cut the central elongated portion 16, creating a widened region 20 near the cut that will fix the anchor 6 relative to the magnet 4. Additionally or alternatively, a crimp may be placed around the central elongated portion 16, positioned adjacent to the magnet 4, and crimped around the portion 16 before cutting the portion 16. In yet another example, a blind or pop rivet mechanism may be used to anchor the magnet 4 to the stomach wall.

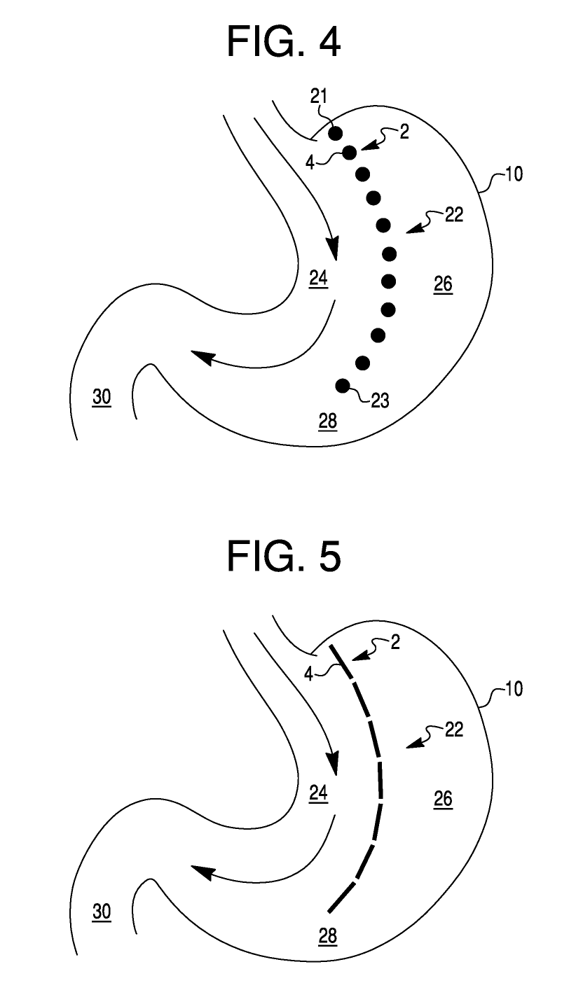

FIGS. 4-8 illustrate exemplary arrangements of magnetic devices 2 secured to stomach wall tissue 10. The anterior portion of the stomach is not shown in these diagrams to allow visualization of the magnetic devices 2 secured to the posterior wall of the stomach. In these examples, a first group of magnetic devices 2 may be secured to the posterior wall of the stomach, and a second group (not shown) of magnetic devices 2 may be secured to an anterior wall of the stomach. The magnets 4 of the magnetic devices 2 may be in an interior of a stomach once secured to stomach wall tissue. The two groups of magnets may pull the anterior and posterior walls of the stomach towards each other, with magnetically joined pairs of magnets 4 creating a restriction within the stomach.

In one example, the two groups of magnetic devices 2 may include the same number of magnets and may mirror each other so that the magnets 4 in the first group align with and are magnetically attracted to corresponding magnets 4 in the second group. In other words, there may be a one-to-one correspondence between magnets 4 in the first group and magnets 4 in the second group. Alternatively, one group may include a different number of magnets 4 than the other group, or one group may include differently shaped magnets 4 than the other group. For example, one group may include a larger number of small round magnets, while the other group may include a smaller number of elongated rectangular magnets so that multiple small magnets (e.g., two, three, four, or more) may align with and magnetically join a single elongated magnet. Additionally or alternatively, each group of magnets may include magnets 4 having different shapes.

The restrictions shown in FIGS. 4-8 may be reversible because the magnetic devices 2 can be removed during a subsequent procedure. Additionally or alternatively, the anchors 6 may be bioabsorbable. In either case, the stomach may be returned to its natural state at a later date using a minimally invasive procedure, or in the case of bioabsorbable anchors 6, with no additional procedure required.

The strength of the magnets 4 used in the arrangements of FIGS. 4-8 may be selected to avoid damage to stomach wall tissue 10. In some current procedures to restrict the stomach, eating too much or other activities may exert too much pressure on the sutures or staples within the stomach and may cause stomach tissue to perforate. However, the strength of the magnetic force between magnetically joined magnets 4 may be selected so that the magnets 4 will pull apart from each other before the anchors 6 pull out of the stomach wall tissue 10. The ability of the magnets 4 to pull apart from each other may avoid potential damage to tissue 10.

Each magnetic device 2 may be a discrete device that is either separate from or separable from other magnetic devices (e.g., not directly connected by a material to other magnetic devices). However, each discrete magnetic device 2 may be magnetically joined at times (e.g., once implanted) to other magnetic devices through a magnetic force, or indirectly connected through stomach tissue. In one example, each of the magnets 4 in a first group is spaced apart from other magnets in the first group once it is secured to stomach wall tissue, and each of the magnets 4 in a second group is spaced apart from other magnets in the second group once it is secured to stomach wall tissue. Providing space between adjacent magnets in a group may allow gastric fluids or food to leak through the spaces between magnetically joined pairs of magnets 4. Allowing gastric fluids to at least partially travel through a restriction created by magnets 4 may reduce acid buildup and may help prevent the formation of ulcers.

The arrangements of FIGS. 4-8 may direct the flow of food to promote satiety. For example, as will be described further below, certain examples may direct food towards regions of the stomach that may regulate hormones when stretched (e.g., the fundus).

Referring to FIG. 4, a first group 22 of magnetic devices 2 may be arranged in a curved pattern that extends from a proximal first end 21 adjacent the fundus to a distal second end 23 near the antrum. A second group of similarly arranged magnets may be secured to an anterior wall of the stomach. The arrangement of FIG. 4 may mimic a sleeve gastrectomy procedure, as it reduces the effective volume of the stomach by creating a restriction dividing the stomach. The magnetic devices 2 may divide the stomach into a first region 24 for the flow of food, indicated by arrows, and a second region 26 substantially blocked from the flow of food. Food may flow from the esophagus, into the first region 24, and towards the intestine 30. The second region 26 may include the fundus and the greater curvature. Accordingly, the arrangement of FIG. 4 may at least partially block food from contacting the fundus and the greater curvature. A space 28 may be provided between the distal second end 23 (e.g., the distal-most pair) of the magnetic devices 2 and a distal wall of the stomach (e.g., the wall of the antrum) to allow gastric fluids to flow from second region 26 to the intestine 30. The space 28 may be greater than a space between the proximal-most pair of magnetic devices 2 at the proximal first end 21 and a proximal wall of the stomach (e.g., a wall of the fundus). In an alternative example, one or more pairs of additional magnetic devices 4 may be secured to the stomach in space 28 to close the space 28.

FIG. 5 illustrates a similar arrangement as FIG. 4, except the magnets 4 of magnetic devices 2 are illustrated having an elongated rectangular shape. As described above, the magnets 4 may be any shape. Providing less space between adjacent magnetic devices 2, as shown in FIG. 5 relative to FIG. 4, may reduce the transfer of food and gastric juices between regions 24 and 26 of the stomach. In one example, there is no space between adjacent magnetic devices 2. The surface area of magnets 4 may affect the strength of the magnetic force between magnets 4 of the first group 22 and magnets 4 of the corresponding second group (not shown) of magnetic devices 2 secured to the anterior wall of the stomach. A stronger force may increase the amount of pressure in the stomach that is required to pull apart the restriction created by magnetically-joined magnets 4. Similar to FIG. 4, the arrangement of FIG. 5 may include a space 28 between a second end of the arrangement, near the antrum, and a distal wall of the stomach. In another example, the magnetic devices 2 may extend closer to the distal wall of the stomach to close the space 28.

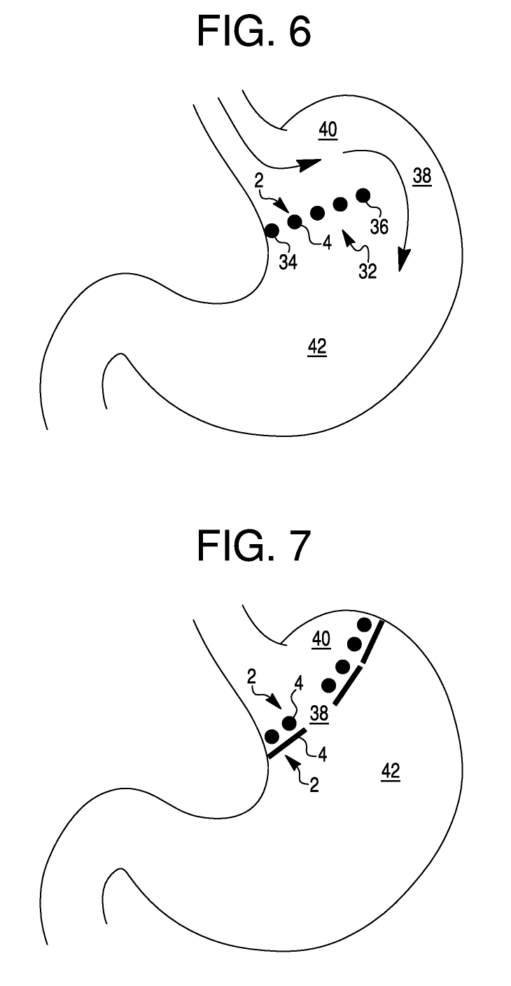

Referring to FIG. 6, a restriction may include a first group 32 (and a corresponding second group, not shown) of magnetic devices 2 extending in a row from a first end 34 adjacent a wall of the lesser curvature of the stomach, towards a wall of the greater curvature of the stomach, to a second end 36. This example may mimic a gastric band, as food is directed through a restricted space 38 provided between the second end 36 of the row of magnetic devices 2 and the wall of the greater curvature. The magnetic devices 2 may divide the stomach into proximal region 40 and distal region 42. Food, indicated by arrows, may flow through the restricted space 38 to travel from the proximal region 40 to the distal region 42. This example may promote satiety by directing food towards the fundus. Stretching the fundus by food may induce a feeling of fullness. When the stomach is empty, ghrelin, which drives hunger, may be secreted. When the wall of the stomach is distended, however, ghrelin secretion may be reduced or stopped.

The magnetic devices 2 of FIG. 6 may be arranged in any configuration that creates a restricted space 38 in a proximal region of the stomach. For example, as shown in FIG. 7, magnetic devices 2 may be secured to the stomach on both sides of a restricted space 38. Magnetic devices 2 may extend in a first row from the lesser curvature towards space 38 and, from the opposite direction, magnetic devices 2 may extend in a second row from the greater curvature towards space 38. In the example shown in FIG. 7, both round and rectangular magnets 4 may be used to create the desired blockage of food and the desired force required to pull apart magnetically joined magnets 4. The round magnets 4 may lie in first and second rows proximal to and parallel to the first and second rows of rectangular magnets. In another example, the round magnets 4 may lie in first and second rows distal to and parallel to the first and second rows of rectangular magnets.

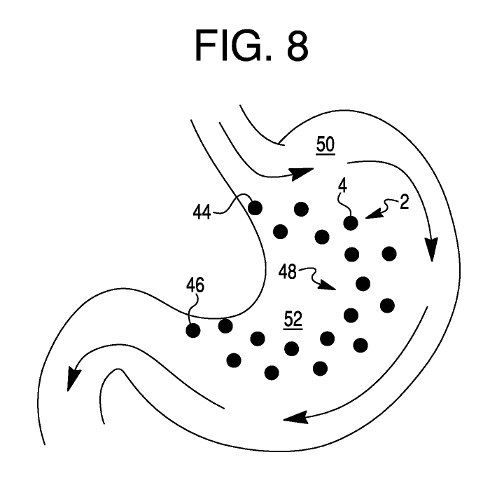

Referring to FIG. 8, another arrangement of magnetic devices 2 may include a plurality of magnetic devices 2 arranged in a curved configuration extending from a first end 44 adjacent a proximal wall of the lesser curvature to a second end 46 adjacent a distal wall of the lesser curvature. The first group 48 of magnets 4 may be secured to a posterior wall of the stomach, and a second group (not shown) may be secured to an anterior wall of the stomach. The curved configuration may be C-shaped. This example may promote satiety by directing food towards the fundus, allowing it to stretch. When stretched by food, the fundus may induce a feeling of fullness by regulating the release of certain hormones (e.g., ghrelin). As illustrated in FIG. 8, the magnetic devices 2 may be generally arranged in two curved rows. Alternatively, the magnetic devices 2 may be arranged in a single row or in an irregular manner following a generally curved path. The magnets 4 may be any shape, such as round or rectangular, and may include a combination of shapes.

The magnetic devices 2 of FIG. 8 may divide the stomach into a first region 50 for the flow of food and a second region 52 generally blocked from the flow of food. However, some food and gastric fluids may travel between adjacent pairs of magnetically-joined magnetic devices 2 to prevent trapping acids in region 52. The example of FIG. 8 may have similar benefits as the examples shown in FIGS. 4 and 5 in that the effective volume of the stomach is reduced in both examples, and food travels in a narrowed path from the esophagus to the intestine. However, the arrangement of FIG. 8, similar to the arrangement of FIG. 6, may direct food towards the fundus to promote satiety.

While principles of the present disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A method of restricting a stomach of a patient, comprising:
transorally inserting a first plurality of magnets and a second plurality of magnets into the stomach of the patient, wherein the magnets of the first plurality are magnetically attracted to the magnets of the second plurality;
securing each magnet of the first plurality of magnets to a discrete location on an interior of a first wall of the stomach, and securing each magnet of the second plurality of magnets to a discrete location on an interior of a second wall of the stomach; and
allowing a plurality of pairs of magnets, each pair including a magnet of the first plurality of magnets and a magnet of the second plurality of magnets, to magnetically join together to create a restriction in the stomach.

2. The method of claim 1, wherein each magnet of the first plurality of magnets and each magnet of the second plurality of magnets is secured to the respective wall of the stomach by a different bioabsorbable anchor.

3. The method of claim 1, wherein the plurality of pairs are arranged in a curved configuration extending from a proximal end adjacent a fundus to a distal end closer to an antrum than the proximal end.

4. The method of claim 3, further comprising providing a distal space between the distal end and a wall of the antrum, wherein the distal space is greater than a proximal space between the proximal end and a wall of the fundus.

5. The method of claim 1, wherein the magnetically-joined plurality of pairs of magnets at least partially block food from a region of the stomach that includes the fundus and the greater curvature.

6. The method of claim 1, wherein the plurality of magnetically-joined pairs of magnets is configured to allow at least some food to pass between adjacent pairs of magnets.

7. The method of claim 1, wherein the plurality of pairs is arranged in a linear configuration extending from a first end adjacent a wall of a lesser curvature of the stomach to a second end closer to a greater curvature of the stomach than the first end.

8. The method of claim 7, further comprising providing a first space between the second end and the greater curvature, wherein the first space is greater than a second space between the first end and the wall of the lesser curvature.

9. The method of claim 1, wherein the plurality of pairs is arranged in a curved configuration extending from a first end adjacent a proximal wall of a lesser curvature of the stomach and a second end adjacent a distal wall of the lesser curvature.

10. A method of restricting a stomach of a patient, comprising:
transorally inserting a plurality of magnets into the stomach of the patient; and
securing each magnet of the plurality of magnets to an interior of the stomach with an anchor, wherein each magnet is secured with a different anchor;
wherein a first portion of the plurality of magnets is secured to a first wall of the stomach, and a second portion of the plurality of magnets is secured to a second wall of the stomach, and the first and second portions are configured to magnetically join to create a restriction that guides food from a distal end of an esophagus towards a fundus.

11. The method of claim 10, wherein the first and second portions are configured to separate to relieve pressure in the stomach.

12. The method of claim 10, wherein the restriction extends in a linear configuration from a first end adjacent a wall of a lesser curvature of the stomach to a second end closer to a greater curvature of the stomach than the first end.

13. The method of claim 10, wherein the restriction extends in a curved configuration from a first end adjacent a proximal wall of a lesser curvature of the stomach to a second end adjacent a distal wall of the lesser curvature.

14. The method of claim 10, wherein the restriction is configured to allow at least some food to pass through the restriction.

15. The method of claim 10, wherein each magnet of the plurality of magnets includes a circular shape.

16. A method of restricting a stomach of a patient, comprising:
inserting a plurality of discrete magnets through an esophagus and into the stomach of the patient; and
securing each of the plurality of magnets to a wall of the stomach, wherein each magnet is magnetically attracted to at least one other magnet;
wherein the magnets are arranged in at least a first group and a second group, with adjacent magnets in the first group spaced apart from each other and with adjacent magnets in the second group spaced apart from each other, and
wherein the first group and the second group are configured to move towards each other through magnetic attraction and to separate from each other to relieve a pressure in the stomach.

17. The method of claim 16, wherein each of the plurality of discrete magnets is secured to an interior of the stomach.

18. The method of claim 16, wherein the first group includes the same number of magnets as the second group.

19. The method of claim 16, wherein each magnet of the plurality of magnets is secured to the wall of the stomach by an anchor that is configured to release the magnet from the wall of the stomach after a predetermined period of time.

20. The method of claim 16, wherein, when the first group and the second group move towards each other, the stomach is divided into a first elongated region for a flow of food, the first elongated region having a proximal end adjacent an esophagus and a distal end adjacent an intestine, and a second region at least partially blocked from the flow of food.
